(19) United States
(12) Patent Application Publication
(10) Pub. No.: US 2023/0399345 A1
(43) Pub. Date: Dec. 14, 2023

(54) CRYSTALLIZED FORM OF LURBINECTEDIN AND METHOD OF MAKING THE SAME

(71) Applicant: PHARMA MAR, S.A., Madrid (ES)

(72) Inventors: Maria Del Mar Zarzuelo Alba, Madrid (ES); María de la Concepción Polanco Noain, Madrid (ES); Sonia Manzanaro López, Madrid (ES); Honorio Velasco, Madrid (ES)

(21) Appl. No.: 18/456,385

(22) Filed: Aug. 25, 2023

Related U.S. Application Data
(63) Continuation of application No. 17/777,985, filed on May 18, 2022.

(30) Foreign Application Priority Data
Nov. 21, 2019 (EP) 19383025.4
May 29, 2020 (WO) PCT/EP2020/065093

(51) Int. Cl. C07D 515/22; A61K 9/19

(52) U.S. Cl. CPC C07D 515/22; A61K 9/19; C07B 2200/13

(57) ABSTRACT

The present invention relates to form B of lurbinectedin of the formula:

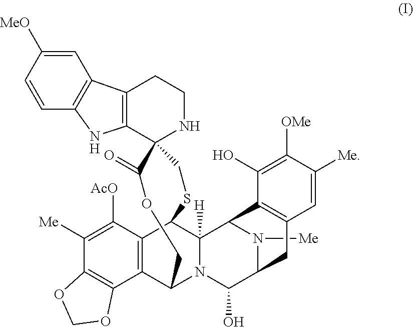 [SMILES: COc1ccc2[nH]c3c(c2c1)CCN[C@]31CS[C@@H]2c3c(OC(C)=O)c(C)c4c(c3[C@H](COC1=O)N1[C@@H]2[C@H]2c3c(cc(C)c(OC)c3O)C[C@@H]([C@@H]1O)N2C)OCO4]

### FIELD OF THE INVENTION

The present invention relates to a novel solid state form of lurbinectedin, to methods for its preparation, and to said form of lurbinectedin for use as a medicament. In addition, the present invention relates to pharmaceutical compositions comprising said form of lurbinectedin, and to methods for the manufacture of pharmaceutical compositions that employ it.

### BACKGROUND OF THE INVENTION

Lurbinectedin, also known as PM01183 and initially called tryptamicidin, is a synthetic antitumoral compound that is currently in clinical trials for the treatment of cancer. The chemical structure of lurbinectedin is represented by formula (I):

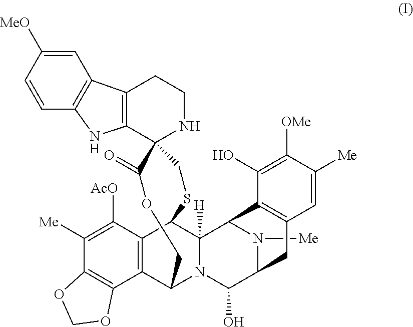 [SMILES: COc1ccc2[nH]c3c(c2c1)CCN[C@]31CS[C@@H]2c3c(OC(C)=O)c(C)c4c(c3[C@H](COC1=O)N1[C@@H]2[C@H]2c3c(cc(C)c(OC)c3O)C[C@@H]([C@@H]1O)N2C)OCO4]

Lurbinectedin has demonstrated highly potent in vitro activity against solid and non-solid tumour cell lines as well as significant in vivo activity in several xenografted human tumor cell lines in mice, such as those for breast, kidney and ovarian cancer. Lurbinectedin exerts its anticancer effect through the covalent modification of guanines in the DNA minor groove that eventually give rise to DNA double-strand break, S-phase arrest and apoptosis in cancer cells.

Patent application WO 03/014127 describes lurbinectedin, pharmaceutical compositions comprising the same and methods of treatment of cancer comprising its administration. In this patent application, lurbinectedin was obtained by reacting compound 1 with water in presence of silver nitrate (Scheme 1) followed by conventional chromatographic purification.

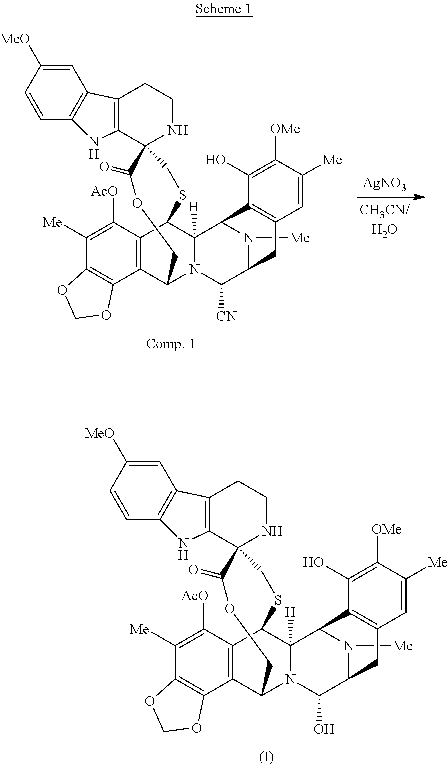

Synergistic combinations of lurbinectedin with other antitumoral drugs are disclosed in WO 2012/062920. Information regarding its mechanism of action and in vivo efficacy can be found in 100^{th} AACR Annual Meeting, Apr. 18-22, 2009, Denver, CO, Abstract Nr. 2679 and Abstract Nr. 4525; and Leal J F M et. al. British J. Pharmacol. 2010, 161, 1099-1110.

Further information regarding the clinical development of PM01183 can be found in:
Elez, M E et. al. Clin. Cancer Res. 2014, 20(8), 2205-2214;
51^{th} ASCO Annual Meeting, May 29-Jun. 2, 2015, Chicago, IL, Abstract Nr. TPS2604 and Abstract Nr. 7509, published in J. Clin. Oncol. 33, 2015 (suppl);
50^{th} ASCO Annual Meeting, May 30-Jun. 3, 2014, Chicago, IL, Abstract 5505;
39^{th} ESMO Congress, Sep. 26-30, 2014, Madrid, Spain, published in Ann. Oncol, 2014, 25(Suppl. 4), page 146 Abs No. 482P; and
26^{th} EORTC-NCI-AACR Symposium on Molecular Targets and Cancer Therapeutics; Nov. 18-21, 2014, Barcelona, Spain, published in Eur. J. Cancer 2014, 50 (Suppl. 6), pages 13-14, Abs. No. 23.

All these disclosures are silent as to the preparation and the nature of specific crystal forms of lurbinectedin.

Polymorphism is a phenomenon relating to the occurrence of different crystal forms for one molecule. There may be several different crystalline forms of the same molecule with distinct crystal structures and varying in physical properties like melting point, XRPD pattern and FTIR spectrum. These polymorphs are thus distinct solid forms which share the molecular formula of the compound from which the crystals are made up, however they may have distinct physical properties such as e.g. chemical stability, physical stability, processability, hygroscopicity, solubility, dissolution rate, bioavailability etc.

The form of lurbinectedin obtained by the process described in WO 03/014127, in the following named form A of lurbinectedin, is amorphous and becomes electrically charged during its manipulation causing production problems. Therefore there is the need to obtain a form of lurbinectedin easier to handle under typical pharmaceutical processing conditions.

### SUMMARY OF THE INVENTION

The inventors of the present invention have found a novel solid state form of lurbinectedin that is easier to handle under typical pharmaceutical processing conditions than the known amorphous form A.

In one aspect the invention relates to a novel solid state form of lurbinectedin, in the following named form B of lurbinectedin. Form B shows advantageous physical properties compared to the known form A.

Form B shows significantly improved triboelectric properties over existing known forms of lurbinectedin. Triboelectric charging is the process by which certain materials become electrically charged after contact with a different material through friction. In many pharmaceutical operations uncontrolled static electricity can cause serious production problems. These problems may include product contamination, product loss, cleaning and safety, and the problems can be exacerbated in a nanomolar cytotoxic drug such as PM01183. Even in the most stringent cleanrooms, static charge attracts particulates from people, processes and equipment, so it is important to take appropriate measures to ensure it is kept to a minimum.

Form B shows a lower average charge density over the known form of lurbinectedin. Form B also shows a narrower dispersion of charge density over the known form of lurbinectedin.

Form B of lurbinectedin has lower residual solvents over the known form of lurbinectedin. Form B also has a simplified impurity profile compared to the known form of lurbinectedin. These characteristics make it especially suitable for the preparation of a medicament.

In a further aspect, the present invention relates to a process for preparing form B of lurbinectedin comprising:
a) preparing an acidic aqueous solution comprising lurbinectedin or a protonated form thereof; and
b) basifying the resulting acid aqueous solution with a base or a basic buffer to precipitate form B of lurbinectedin.

The form B of lurbinectedin may be subsequently converted into a different physical form, preferably an amorphous form.

In a further aspect, the present invention relates to pharmaceutical compositions comprising form B of lurbinectedin and a pharmaceutically acceptable carrier.

In a further aspect, the present invention relates to a pharmaceutical composition comprising lurbinectedin manufactured via form B of lurbinectedin and a pharmaceutically acceptable carrier.

In a further aspect, the present invention relates to form B of lurbinectedin for use in the manufacture of a pharmaceutical composition comprising lurbinectedin.

In a further aspect, the present invention relates to the use of form B of lurbinectedin in the manufacture of a pharmaceutical composition comprising lurbinectedin.

In a further aspect, the present invention relates to form B of lurbinectedin for use as a medicament.

In a further aspect, the present invention relates to compositions comprising form B of lurbinectedin and a pharmaceutically acceptable carrier for use as a medicament.

In a further aspect, the present invention relates to form B of lurbinectedin for use as a medicament for the treatment of cancer.

In a further aspect, the present invention relates to compositions comprising form B of lurbinectedin and a pharmaceutically acceptable carrier for use as a medicament for the treatment of cancer.

In a further aspect, the present invention relates to processes for the manufacture of pharmaceutical compositions comprising lurbinectedin that employ form B of lurbinectedin, preferably as starting material.

In a further aspect, the present invention is also directed to the use of form B of lurbinectedin, or of a pharmaceutical composition comprising form B of lurbinectedin and a pharmaceutically acceptable carrier in the treatment of cancer, or in the preparation of a medicament for the treatment of cancer. Other aspects of the invention are methods of treatment, and form B of lurbinectedin for use in these methods. Therefore, the present invention further provides a method of treating any mammal, notably a human, affected by cancer which comprises administering to the affected individual a therapeutically effective amount of form B of lurbinectedin or of a pharmaceutical composition comprising form B of lurbinectedin and a pharmaceutically acceptable carrier.

The present invention further provides a method of treating any mammal, notably a human, affected by cancer which comprises administering to the affected individual a therapeutically effective amount of lurbinectedin which has been manufactured via form B of lurbinectedin; or of a pharmaceutical composition comprising lurbinectedin which has been manufactured via form B of lurbinectedin and a pharmaceutically acceptable carrier.

In a further aspect, the present invention relates to lurbinectedin having residual solvents of not more than 1%, 0.5%, 0.1% or substantially not detected.

In a further aspect, the present invention relates to lurbinectedin having a water content of above 1.6% w/w, or of 1.7-5% w/w.

In a further aspect, the present invention relates to partially crystalline lurbinectedin.

In a further aspect, the present invention relates to a pharmaceutical composition or a pharmaceutical intermediate comprising partially crystalline lurbinectedin as defined herein.

In a further aspect, the present invention relates to a pharmaceutical compositions made from a process including partially crystalline lurbinectedin as defined herein.

In a further aspect, the present invention relates to a process for the manufacture of a lurbinectedin composition, said process employing lurbinectedin as defined herein, or partially crystalline lurbinectedin as defined herein; preferably as a starting material.

In a further aspect, the present invention relates to a lurbinectedin infusion solution, a reconstituted solution, a lyophilized composition or a bulk composition according to the processes as defined herein.

In a further aspect, the present invention relates to partially crystalline lurbinectedin as defined herein, for use as a medicament, for use in the manufacture of a medicament or for use in the manufacture of a medicament for the treatment of cancer.

In a further aspect, the present invention relates to a method of treating an individual affected by cancer comprising administering to said affected individual a therapeutically effective amount of partially crystalline lurbinectedin as defined herein.

### BRIEF DESCRIPTION OF THE DRAWINGS

FIG. 1: X-ray powder diffractogram (XRPD) of form A of lurbinectedin (Batch R05).

FIG. 2a: X-ray powder diffractograms (XRPD) of two batches of form B of lurbinectedin (Batches 1924128-LT (overlaid) and 1924129-LT).

FIG. 2b: X-ray powder diffractograms (XRPD) of form B of lurbinectedin made by mixing 15 mg Batch 1711182-2 (form B partly crystalline) and 15 mg Batch P02 (amorphous) with 1 ml water. The suspension was stirred at r.t. for 24 hours. The resulting solid was filtered off).

FIG. 3: TG-FTIR of form B of lurbinectedin (Batch 1711182-2).

FIG. 4: DSC of form B of lurbinectedin (Batch 1711182-2).

FIG. 5: DVS of form B of lurbinectedin (Batch P05).

FIG. 6: Superimposed XRPD patterns of form B of lurbinectedin in an initial 1:1 mixture of forms A and B of lurbinectedin, after 6 h of phase equilibration in water and after 24 h of phase equilibration in water, from top to bottom. (Mixtures were prepared mixing form A of lurbinectedin (batch P02) and Form B of lurbinectedin (batch 1711182-2)).

FIG. 7a: IR of form A of lurbinectedin (Batch P04).

FIG. 7b: IR of form B of lurbinectedin (Batch 1711182-2).

FIG. 8: Scheme of the Faraday cage.

FIG. 9a: Electrostatic charge (nC) of different amounts of form A of lurbinectedin (Batch P04) and form B of lurbinectedin (Batch 1924129-LT).

FIG. 9b: Electrostatic charge (nC) of different amounts of form A of lurbinectedin (Batch R05) and form B of lurbinectedin (Batch 1924128-LT)

FIG. 10a: Charge density of form A of lurbinectedin (Batch P04) and form B of lurbinectedin (Batch 1924129-LT).

FIG. 10b: Charge density of form A of lurbinectedin (Batch R05) and form B or lurbinectedin (Batch 1924128-LT).

### DETAILED DESCRIPTION OF THE INVENTION

As used herein the term “room temperature” indicates that the applied temperature is not critical and that no exact temperature value has to be kept. Usually “room temperature” is understood to mean temperatures of about 15° C. to about 25° C. [see. e.g. EU Pharmacopoeia 7.2, 1.2 (2011)].

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

Alkanes in the present invention may be branched or unbranched, and have from about 5 to about 10 carbon atoms. One more preferred class of alkanes has from 5 to 9 carbon atoms. Even more preferred are alkanes having 5, 6 or 7 carbon atoms. Particularly preferred alkanes of this invention are n-pentane, n-hexane, n-heptane, cyclohexane, and methylcyclohexane. As used herein, the term alkane, unless otherwise stated, refers to both cyclic and noncyclic alkanes.

Pharmaceutically acceptable solvents in the context of the present invention are those classified under classes 2 and 3 of the guideline “Impurities: Guideline for residual solvents Q3C(R6)” of the International Conference on Harmonisation of Technical Requirements for Registration of Pharmaceuticals for Human Use.

In an embodiment, the present invention relates to form B of PM01183.

Form B of lurbinectedin can be characterized by showing an X-ray powder diffractogram pattern comprising four or more characteristic peaks at 2-theta angles selected from 6.2±0.2°, 7.6±0.2°, 9.0±0.2°, 10.9±0.2°, 14.9±0.2° and 15.3±0.2°. Form B may alternatively be characterized by showing an X-ray powder diffractogram pattern comprising five or more of said characteristic peaks. Alternatively, Form B may be characterized by showing an X-ray powder diffractogram pattern comprising all six of said characteristic peaks.

Particularly, Form B of lurbinectedin can be characterized by an X-ray powder diffractogram pattern comprising peaks and intensities as shown in the following table:

| Angle | Relative intensity |
|---|---|
| [2-theta] | [%] |
| 6.2 ± 0.2° | 79 ± 6 |
| 7.6 ± 0.2° | 100 ± 3 |
| 9.0 ± 0.2° | 63 ± 3 |
| 10.9 ± 0.2° | 100 ± 3 |
| 14.9 ± 0.2° | 76 ± 3 |
| 15.3 ± 0.2° | 75 ± 3 |

In a preferred embodiment further peaks may be found at 2-theta angles of 12.4±0.2°, 19.2±0.2° and 26.5±0.2°. Particularly, Form B of lurbinectedin can be characterized by an X-ray powder diffractogram pattern comprising characteristic peaks and intensities as shown in the following table:

| Angle | Relative intensity | Angle | Relative intensity |
|---|---|---|---|
| [2-theta] | [%] | [2-theta] | [%] |
| 6.2 ± 0.2° | 79 ± 6 | 14.9 ± 0.2° | 76 ± 3 |
| 7.6 ± 0.2° | 100 ± 3 | 15.3 ± 0.2° | 75 ± 3 |
| 9.0 ± 0.2° | 63 ± 3 | 19.2 ± 0.2° | 34 ± 3 |
| 10.9 ± 0.2° | 100 ± 3 | 26.5 ± 0.2° | 33 ± 3 |
| 12.4 ± 0.2° | 40 ± 3 | | |

In a more preferred embodiment, further peaks may be found at 2-theta angles of 18.4±0.2°, 20.7±0.2° and 24.9±0.2°. Particularly, Form B of lurbinectedin can be characterized by an X-ray powder diffractogram pattern comprising characteristic peaks and intensities as shown in the following table:

| | Relative | | Relative |
|---|---|---|---|
| Angle | intensity | Angle | intensity |
| [2-theta] | [%] | [2-theta] | [%] |
| 6.2 ± 0.2° | 79 ± 6 | 15.3 ± 0.2° | 75 ± 3 |
| 7.6 ± 0.2° | 100 ± 3 | 18.4 ± 0.2° | 29 ± 3 |
| 9.0 ± 0.2º | 63 ± 3 | 19.2 ± 0.2° | 34 ± 3 |
| 10.9 ± 0.2° | 100 ± 3 | 20.7 ± 0.2º | 32 ± 3 |
| 12.4 ± 0.2° | 40 ± 3 | 24.9 ± 0.2º | 26 ± 3 |
| 14.9 ± 0.2° | 76 ± 3 | 26.5 ± 0.2° | 33 ± 3 |

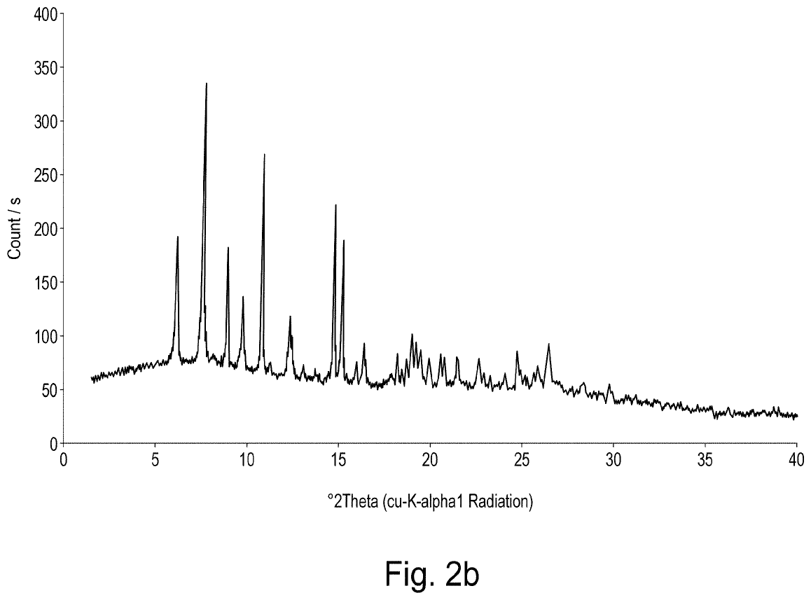

In a most preferred embodiment, the present invention relates to form B of lurbinectedin that exhibits an X-ray powder diffraction pattern substantially the same as any one of the X-ray powder diffraction patterns shown in FIG. 2a or 2b.

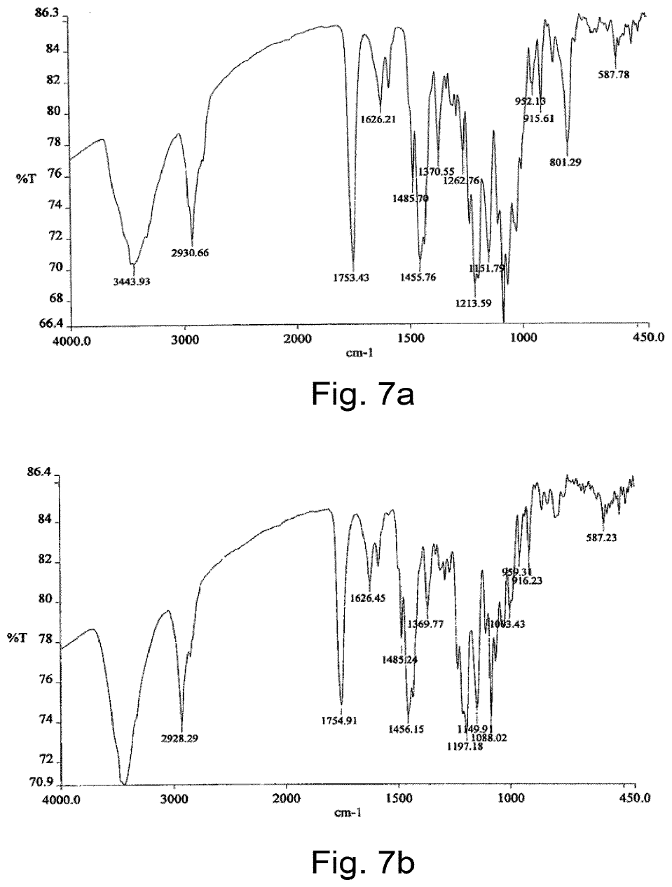

In addition, Form B of lurbinectedin can be characterized by showing an IR spectrum comprising peaks at wavelengths of 2928, 1755, 1626, 1485, 1456, 1370, 1197, 1150, 1088, 1003, 959, 916, and 587. An illustrative IR spectrum is displayed in FIG. 7b.

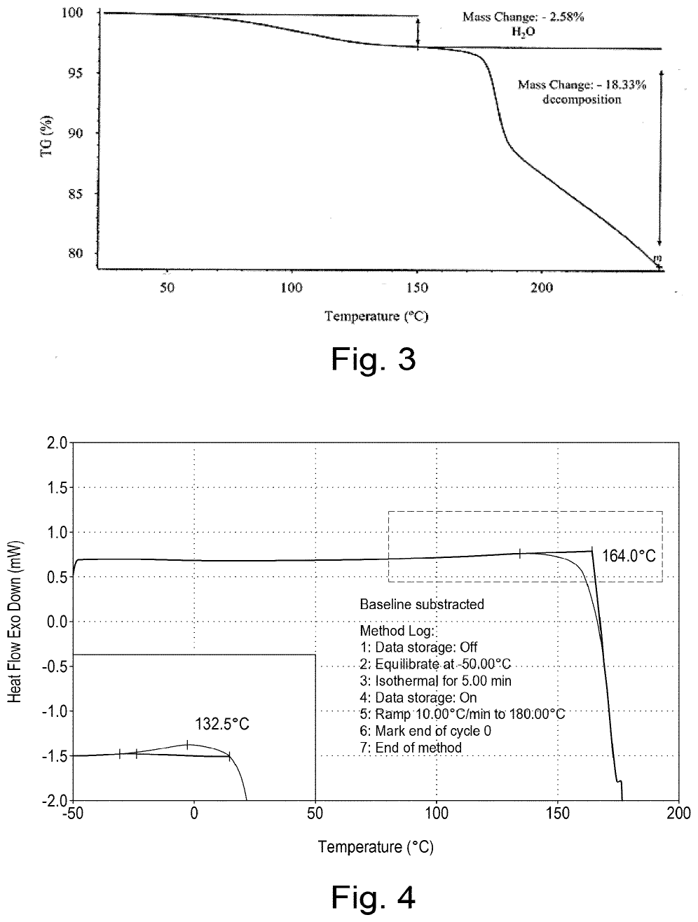

In addition, Form B of lurbinectedin can be characterized by TG-FTIR degradation above 150° C. Alternatively, or in addition, Form B of lurbinectedin can be characterized by a TG-FTIR mass change to 150° C. being due to the loss of water. The loss due to water may be less than about 5%, less than about 4%, or less than about 3%. Alternatively, or in addition, Form B of lurbinectedin can be characterized by TG-FTIR indicating a loss of water, preferably around 2-3% water, more preferably 2.6% water. An illustrative TG-FTIR is displayed in FIG. 3.

In addition, Form B of lurbinectedin can be characterized by DSC wherein degradation begins above 130° C. An illustrative DSC thermogram is displayed in FIG. 4.

In an embodiment, form B of lurbinectedin has an average charge density of not more than about 30 nC/g, not more than about 20 nC/g, not more than about 10 nC/g, not more than about 6 nC/g, not more than about 5 nC/g, about 5±2 nC/g, about 4±2 nC/g, about 4-5 nC/g, about 5 nC/g, or about 4 nC/g.

In an embodiment, form B of lurbinectedin has a dispersion of charge density of less than 4.8 nC/g, of between about 0.7 nC/g to less than 4.8 nC/g, or 2.4±2 nC/g.

In an embodiment, form B of lurbinectedin has a water content of above 1.6% w/w, or of 1.7-5% w/w.

In an embodiment, form B of lurbinectedin has residual solvents of not more than 1%, 0.5%, 0.1% or substantially not detected.

The present invention encompasses lurbinectedin comprising at least a detectible amount of form B, up to 1% form B, up to 5% form B, up to 10% form B, up to 50% form B, up to 90% form B, or be substantially pure form B.

In an embodiment, the invention relates to a process for preparing form B of lurbinectedin comprising:
a) preparing an acidic aqueous solution comprising lurbinectedin or a protonated form thereof; and
b) basifying the resulting acid aqueous solution with a base or a buffer to precipitate form B of lurbinectedin.

In step a), a solution of lurbinectedin in acid water is provided. Examples of methods for preparing such solution include, but are not limited to:
dissolving any solid form of lurbinectedin in acidic water; and
extracting lurbinectedin from a solution comprising lurbinectedin in a water-immiscible organic phase to acidic water.

In a preferred embodiment the acidic aqueous solution of lurbinectedin is obtained by dissolving lurbinectedin in acidic water.

Any form of lurbinectedin may be applied e.g. amorphous lurbinectedin.

The concentration of lurbinectedin in acid water may range from about 10 to about 50 g/L. Particularly preferred are concentrations from about 15 to about 40 g/L, being more preferred concentrations from about 20 to about 30 g/L. Most preferred concentration of lurbinectedin in acid water is about 26 g/L.

The preferred pH of the acid water may range from about 1 to about 4, more preferably from about 1 to about 3, even more preferably from about 1 to about 2 and most preferably is about 1. The acid condition may be provided by an acid or by a buffer. Suitable pharmaceutically acceptable acids include hydrochloric acid, phosphoric acid, sulfuric acid, carboxylic acids such as aliphatic and aromatic carboxylic acids. More preferred acids include hydrochloric acid, phosphoric acid, sulfuric acid, trifluoroacetic acid, nitrobenzoic acid and citric acid. Suitable acid buffering agents provide a pH between about 1 to about 4. Examples of suitable acid buffering agents include phosphate buffer, citrate buffer, lactate buffer, ascorbate buffer, tartaric/citrate buffer, bicarbonate/hydrochloric acid buffer, acetate buffer, succinate buffer and glycine/hydrochloric acid buffer. More preferably the acid condition is provided by an acid and most preferably the acid is hydrochloric acid.

The preferred pH of the solution of lurbinectedin in acidic water may range from about 1 to about 4, from about 1 to about 3, or about 2 to about 3.

In step b), the resulting acid aqueous solution is treated with an excess of base or buffer to basify it and precipitate form B of lurbinectedin.

The basification may be carried out with a base or with a buffer. The preferred pH of the resulting basic solution may range from about 8 to about 11, most preferably from about 9 to about 11. Suitable pharmaceutically acceptable bases include carbonates, hydroxides, hydrogen carbonates and ammonium salts. Particularly preferred bases are sodium carbonate, potassium carbonate, NH₄OH, lithium hydroxide, sodium hydroxide, potassium hydroxide, sodium hydrogen carbonate and potassium hydrogen carbonate. Suitable basic buffers provide a pH between about 8 to about 11. Examples of suitable basic buffers include ammonium and phosphate buffers such as KH₂PO₄ buffer, Na₂HPO₄/citric acid, and NH₄Cl—NH₄OH. In a preferred embodiment the basification is carried out with a buffer and in a most preferred embodiment the basification is carried out with a NH₄Cl—NH₄OH buffer.

The obtained form B of lurbinectedin can be separated by isolation operations such as filtration or centrifugation, preferably by filtration. Moreover, after separation, the separated solid may be subjected to a drying treatment by any known method. The precipitate can be dried preferably under vacuum at a temperature preferably ranging from about 15 to 35° C., more preferably from about 20 to 30° C., and most preferably at about 25° C. for a time preferably ranging from about 10 to 24 hours, more preferably from about 16 to 20 hours and most preferably for about 18 hours.

In a preferred embodiment the acid aqueous solution obtained after step a) is washed one or more times with a pharmaceutically acceptable, water-immiscible, polar solvent and one or more times with a pharmaceutically acceptable, water-immiscible, non-polar solvent, before treating it with an excess of base or buffer in step b).

Examples of pharmaceutically acceptable, water immiscible, polar solvents suitable for this washing are chloroform, 1-butanol, 2-butanol, butyl acetate, ethyl acetate, methyl acetate, 1-pentanol, propyl acetate and dichloromethane. More preferred pharmaceutically acceptable, water-immiscible, polar solvents for this washing are chloroform, ethyl acetate and dichloromethane, with dichloromethane the most preferred.

Preferred pharmaceutically acceptable, water-immiscible, non-polar solvents suitable for this washing are C5-C7 alkanes such as n-heptane, n-hexane, n-pentane, cyclohexane and methylcyclohexane; being n-pentane the most preferred.

In an embodiment, the present invention relates to pharmaceutical compositions comprising form B of lurbinectedin and a pharmaceutically acceptable carrier.

Examples of pharmaceutical compositions include any solid (tablets, pills, capsules, granules, etc.) or liquid (solutions, suspensions or emulsions) compositions for oral, topical or parenteral administration.

The present invention encompasses pharmaceutical compositions comprising lurbinectedin comprising at least a detectible amount of form B, up to 1% form B, up to 5% form B, up to 10% form B, up to 50% form B, up to 90% form B, or be substantially pure form B. The present invention also encompasses pharmaceutical compositions comprising an effective amount of form B of lurbinectedin and a pharmaceutically acceptable carrier.

In an embodiment, the present invention relates to form B of lurbinectedin for use as a medicament and to a composition comprising form B of PM01183 and a pharmaceutically acceptable carrier for use as a medicament. It is particularly preferred that the medicament is for the treatment of cancer. Particularly preferred types of cancer are selected from sarcomas, including soft tissue sarcomas, breast cancer, ovarian cancer, endometrial cancer and lung cancer, including non-small cell lung cancer and small cell lung cancer.

In an embodiment, the present invention relates to a process for the manufacture of pharmaceutical compositions comprising lurbinectedin that employs form B of lurbinectedin. Preferably form B is employed as a starting material. In a further embodiment, form B is employed or formed at any stage during the manufacturing process.

In a preferred embodiment the process is for the manufacture of pharmaceutical compositions comprising lurbinectedin and a disaccharide.

In a particularly preferred embodiment the process is for the manufacture of lyophilised pharmaceutical compositions comprising lurbinectedin and a disaccharide.

In a more preferred embodiment the process comprises preparing a bulk solution for lyophilizing by dissolving form B of lurbinectedin in an acidic medium, mixing the pre-dissolved lurbinectedin with the other components of the bulking solution and, optionally, adjusting the pH of the final solution.

In a most preferred embodiment, the process further comprises freeze-drying the bulk solution.

Examples of suitable disaccharides for the above mentioned process include lactose, trehalose, sucrose, maltose, isomaltose, cellobiose, isosaccharose, isotrehalose, turanose, melibiose, gentiobiose and mixtures thereof. The most preferred disaccharide is sucrose.

The ratio of lurbinectedin to the bulking agent in embodiments of this embodiment of the invention is determined according to the solubility of the bulking agent and, when the formulation is freeze dried, as according to the freeze dryability of the bulking agent. It is envisaged that this ratio (w/w) can be about 1:1 in some embodiments, while other embodiments illustrate ratios in the range from about 1:10 to about 1:1. It is envisaged that other embodiments have such ratios in the range from about 1:10 to about 1:100 and still further embodiments have such ratios in the range from about 1:100 to about 1:1500. The ratio of PM01183 to bulking agent is typically from about 1:100 to about 1:1500, preferably from about 1:100 to about 1:800, more preferably from about 1:100 to about 1:400, and even more preferably about 1:200.

Embodiments of processes providing compositions that contain lurbinectedin can be made by preparing a bulking solution by dissolving form B of lurbinectedin in acidic medium, mixing the pre-dissolved lurbinectedin with the other components of the bulking solution. Usually the bulk solution will be buffered, for example to a pH of about 4. Suitable buffering agents include phosphate buffer and citrate buffer. Other possible buffers can be used, such as phosphate/citrate buffer (a mixture of phosphate buffer and citrate buffer), lactate buffer, ascorbate buffer, tartaric/citrate buffer, bicarbonate/hydrochloric acid buffer, acetate buffer, succinate buffer and glycine/hydrochloric acid buffer. Mixtures of buffers can be used. Biocompatible buffer that permit the control of pH at a desired value provide additional embodiments of this invention.

Other components can be included in the bulk solution, for example surface-active agents such as polyoxyethylene 20 sorbitan monooleate or polyoxyl 40-stearate. Other possible surface-active agents include phospholipids, such as lecithin; polyoxyethylene-polyoxypropylene copolymers, such as Pluronic surfactant; polyoxyethylene ester of 12-hydroxystearic acid, such as Solutol surfactant, ethoxylates of cholesterol, such as diacyl glycerol, dialkyl glycerol; bile salts, such as sodium cholate, sodium deoxycholate, sucrose esters, such as sucrose monolaurate, sucrose monooleate; polyvinyl pyrrolidone (PVP); or polyvinyl alcohol (PVA).

In a preferred embodiment, the process further comprises the step of freeze-drying the bulking solution.

The formulation obtained by this process is normally supplied as a vial containing the lyophilised product. This supply form, however, is not a limitation of the present invention. To provide a vial containing the lyophilised product, the bulk solution is added to a vial and freeze-dried.

The term “mixtures thereof” and “combinations thereof” as used herein refer to at least two entities that provide the antecedent basis for the term “mixture thereof” or “combinations thereof”. By way of illustration, but not as a limitation, the term “product comprising at least one of A, B, C and mixtures thereof” refers to embodiments of the product for which any of the following is satisfied: A is in the product; B is in the product; C is in the product; A and B are in the product; A and C are in the product; B and C are in the product; and A, B, and C are in the product.

Administration of form B of lurbinectedin, or of a composition comprising it, or of a composition manufactured using form B of lurbinectedin, preferably as starting material, may be by any suitable method, such as intravenous infusion, oral preparations, and intraperitoneal and intravenous administration. Infusion times of up to 24 h are preferred, more preferably 1-12 hours, with 1-6 hours most preferred. Short infusion times which allow treatment to be carried out without an overnight stay in hospital are especially desirable. However, infusion may be 12 to 24 hours or even longer if required. Infusion may be carried out at suitable intervals of say 1 to 4 weeks. Pharmaceutical compositions comprising form B of lurbinectedin or manufactured using form B of lurbinectedin, preferably as starting material may be delivered by liposome or nanosphere encapsulation, in sustained release formulation or by other standard delivery means.

The correct dosage of the compound will vary according to the particular formulation, the mode of application, and the particular situs, host and tumour being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

In addition, form B of lurbinectedin has performance that is greater than that of the form A of lurbinectedin in terms of residual solvents and impurities profile. Accordingly, this form B of lurbinectedin can preferably be used in the manufacture of medicaments.

As used herein, the terms “treat”, “treating” and “treatment” include the eradication, removal, modification, or control of a tumor or primary, regional, or metastatic cancer cell or tissue and the minimization or delay of the spread of cancer.

### Pharmaceutical Compositions and Methods of Preparation

Pharmaceutical compositions of lurbinectedin that can be used include solutions, lyophilized compositions, etc., with suitable excipients for intravenous administration.

In embodiments according to the present invention, the pre-lyophilized lurbinectedin or pre-solution lurbinectedin comprises at least some crystalline material. The pre-lyophilized/pre-solution lurbinectedin may be partially crystalline. The pre-lyophilized/pre-solution lurbinectedin may be a solid state crystalline form as described herein. The pre-lyophilized/pre-solution lurbinectedin may contain Form B as described herein. Using lurbinectedin as defined in the present invention leads to advantages, for example better control of impurities and/or degradation products.

As such, the present invention provides pharmaceutical compositions and their methods of preparation wherein the composition manufacturing process utilized a solid-state form as disclosed herein. In an embodiment, this is Form B.

In embodiments, lurbinectedin is supplied and stored as a stable and sterile lyophilized product comprising lurbinectedin, a buffer derived from an organic acid (e.g. an organic carboxylic acid buffer), a disaccharide, and a sufficient base to provide an appropriate pH for injection when the composition is reconstituted in an appropriate solvent.

In some embodiments, the organic carboxylic acid buffer is derived from an organic acid selected from the group consisting of lactic acid, butyric acid, propionic acid, acetic acid, succinic acid, citric acid, ascorbic acid, tartaric acid, malic acid, maleic acid, fumaric acid, glutamic acid, aspartic acid, gluconic acid, and α-ketoglutaric. In some embodiments, the organic carboxylic acid buffer is derived from an organic acid selected from lactic acid or succinic acid. In some embodiments, the organic carboxylic acid buffer is derived from lactic acid. In certain embodiments, the buffer is not a phosphate buffer.

In some embodiments, the disaccharide is selected from the group consisting of sucrose, trehalose or lactose, or a combination thereof. In some embodiments, the disaccharide is sucrose.

In some embodiments, the base is selected from the group consisting of carbonates, hydroxides, hydrogen carbonates and ammonium salts. Particularly preferred bases are sodium carbonate, potassium carbonate, calcium carbonate, NH₄OH, sodium hydroxide, potassium hydroxide, calcium hydroxide, sodium hydrogen carbonate, potassium hydrogen carbonate and calcium hydrogen carbonate. In some embodiments, the base is sodium hydroxide.

In some embodiments, the pH of the reconstituted lyophilized composition is about 4. In some embodiments, the pH of the reconstituted lyophilized composition is about from about 3 to about 5. In some embodiments, the pH of the reconstituted lyophilized composition is about from about 3.5 to about 4.5. In some embodiments, the pH of the reconstituted lyophilized composition is 3.8 to 4.1.

In some embodiments, the stable lyophilized product comprises lurbinectedin; lactic acid; sodium hydroxide and sucrose and the pH of the reconstituted lyophilized composition is 3.8 to 4.1. In some embodiments, the stable lyophilized product comprises 4 mg lurbinectedin; 22.1 mg lactic acid; 5.1 mg sodium hydroxide (or, including, about mmol lactate); and 800 mg sucrose. In some embodiments, the stable lyophilized product consists essentially of 4 mg lurbinectedin; 22.1 mg lactic acid; 5.1 mg sodium hydroxide (or, including, about 0.25 mmol lactate); and 800 mg sucrose.

The lurbinectedin-containing formulations of this invention can be made by freeze-drying a composition of this invention in the form of a buffered bulk solution including lurbinectedin, a buffer derived from an organic acid, such as a lactate buffer or a succinate buffer, and a disaccharide. The disaccharide is preferably sucrose. Usually the bulk solution will be buffered, for example to a pH of about 3 to 5, preferably about 3.5 to 4.5, more preferably pH 3.8 to 4.1. The preferred buffering agent is a sodium lactate buffer. In preferred embodiments, the lactate buffer comprises lactic acid and a base, preferably an inorganic, pharmaceutically accepted base such as sodium hydroxide.

As such, in embodiments of the present invention there is provided a buffered lyophilized composition including lurbinectedin, a buffer derived from an organic acid, such as a lactate buffer or a succinate buffer, and a disaccharide; wherein the buffer is configured such that upon reconstitution the pH of the reconstituted lyophilized composition is from about 3 to about 5, about 3.5 to about 4.5, or 3.8 to 4.1

The present invention has identified methodologies that allow for complete dissolution of lurbinectedin in desired buffers whilst minimizing impurity generation. In embodiments, the use of an organic acid buffer allows for direct dissolution of lurbinectedin in the organic acid buffer (preferably at pH about 1 to 5, about 2 to 4.5, about 3 to 4.5 or about 4) followed by addition of bulking agent such as disaccharide, preferably sucrose. Such a formulation strategy enables direct dissolution into the bulk formulation and avoids the need for a pre-dissolution step. In an embodiment, there is provided direct dissolution of lurbinectedin, comprising dissolving lurbinectedin in an organic acid buffer (preferably at pH about 1 to 5, about 2 to 4.5, about 3 to 4.5 or about 4), followed by addition of bulking agent such as disaccharide, preferably sucrose, to form a bulk solution. The bulk solution may undergo sterilizing filtration. The bulk solution may then be filled in vials according to the desired dose. The bulk solution in vials may then be lyophilized to form a lyophilized buffered lurbinectedin formulation. The lyophilized formulation may then be reconstituted to form a reconstituted solution. The reconstituted solution may be diluted to form an injection solution. Preferably, with direct dissolution the lurbinectedin is amorphous or substantially amorphous.

As explained herein, lurbinectedin has limited aqueous solubility. It was found that lurbinectedin solubility is improved in the bulk solution by first forming a concentrated pre-solution of the lurbinectedin in a buffer derived from an organic acid, for example lactic acid, succinic acid, citric acid, or acetic acid which is further diluted with water for injection. A disaccharide is then dissolved in an aqueous solution containing a basic ingredient, for example an aqueous sodium hydroxide solution, and upon adjusting the pH to a set value, the pre-solution of the lurbinectedin and the buffer solution containing a disaccharide are mixed to obtain the lurbinectedin bulk solution in an organic buffer, pH=4 containing a disaccharide (for example, sucrose). Following this process, the lurbinectedin concentration can be increased in the bulk solution enabling the vial fill volume to be reduced. In these embodiments of the present invention, the fill volume is usually reduced by about 80% with respect to that of the conventional fill volume. By way of illustration, but not as a limitation, embodiments of this invention provide a fill volume of 1 mg lurbinectedin in 2 ml solution within a 10 ml vial; or 4 mg lurbinectedin in 8 ml solution within a 30 ml vial. The fill volume can optionally be reduced further in other embodiments of this invention by increasing the lurbinectedin concentration.

Provided are processes useful for improving the solubility of lurbinectedin in the bulking solution that comprise dissolving lurbinectedin in lactic acid, for example 0.31M lactic acid (25 mg/mL), and subsequent dilution of the solution with water for injection to yield a lurbinectedin concentrated solution in 0.1M lactic acid, mixing the solution containing pre-dissolved lurbinectedin with a buffer salt solution comprising sodium lactate buffer and a disaccharide, and, optionally, adjusting the pH. In some illustrative, but not limiting, embodiments of this invention, pH adjustment is accomplished with a lactate buffer.

Illustrative embodiments of bulk solution for freeze drying according to the present invention are provided by a solution of lurbinectedin buffered at pH 4 with sodium hydroxide and lactic acid with sucrose as bulking agent.

An illustrative embodiment of the methodology according to this invention provides as follows: lurbinectedin is dissolved in 0.31M lactic acid, pH˜3 and subsequently diluted with water for injection to yield a lurbinectedin concentrated solution of 8.3 mg/mL lurbinectedin in 0.1M lactic acid, pH˜3. Sodium lactate buffer salt solution is prepared by mixing 0.31M lactic acid solution with 0.01M sodium hydroxide solution to create a 0.05M lactate buffer salt solution. Sucrose is then added to the sodium lactate buffer salt solution. The 0.05M lactate buffer salt solution containing sucrose is diluted with water for injection to yield a 0.04M sodium lactate buffer, pH˜4.2 containing 17% sucrose. Both solutions, 8.3 mg/mL lurbinectedin in 0.1M lactic acid, pH˜3 and 0.04M sodium lactate buffer, pH˜4.2 containing 17% sucrose are then mixed. Dissolution is visually checked at all steps before continuing, and dissolution is considered complete when it is so appreciated visually The pH of the solution is checked and adjusted to a value in the range from about 1 to about 5, more preferably in the range from about 2 to about 4.5, even more preferably in the range from about 3 to about 4.5, and most preferably to a pH of about 4.0 by slow addition of a suitable acid or base. A preferred embodiment of such acid is lactic acid, in which case a preferred concentration is about 0.1M. A suitable base is optionally added for pH control. A preferred embodiment of such base is sodium hydroxide, preferably in solution, in which case a preferred concentration is about 0.1M. The volume is finally adjusted by addition of a suitable, biocompatible fluid, preferably water for injection. The resulting bulk solution preferably comprises 0.5 mg lurbinectedin in 0.03M sodium lactate buffer, pH=4, with 10% (w/v) sucrose. The bulk solution is then filled in vials according to the desired dose.

In embodiments, the lurbinectedin to be dissolved is at least partially crystalline. The lurbinectedin to be dissolved may be in the solid state form(s) described herein. Crystalline lurbinectedin (including partially crystalline) lurbinectedin has been found to be less soluble than amorphous lurbinectedin. By way of example, while the direct dissolution of amorphous lurbinectedin at 0.5 mg/mL in 0.03 M sodium lactate buffer pH 4 was completed in approximately 30 minutes, partly crystalline lurbinectedin reached only 60-70% of the target concentration in 2 hours, meaning that it had much slower dissolution kinetics.

It has been found that decreasing the pH accelerates the dissolution kinetics of partly crystalline lurbinectedin. As such, in embodiments, a concentrated lurbinectedin solution is prepared in organic acid before addition of other excipients. In preferred embodiments, the organic acid has a pH less than 4, preferably less than 3.5, more preferably less than 3, or around 3. The maximum solubility of lurbinectedin was investigated in different molarities of the organic acid lactic acid. Solubility was high and increased linearly ranging from 7.2 mg/ml for 0.05M lactic acid to 90.4 mg/ml for 0.5M lactic acid. In a preferred embodiment, lurbinectedin is dissolved in an organic acid with a molarity of around 0.1M to 0.5M, preferably around 0.2M to 0.4M, more preferably around 0.3M organic acid. An exemplary molarity is 0.31M organic acid.

Lurbinectedin may be pre-dissolved in high concentration organic acid. In a preferred embodiment, the pre-dissolution step is at least 30 minutes, at least 60 minutes or at least 90 minutes, between 30-90 minutes, between 60-90 minutes, between 60-70 minutes or around 60 minutes. Following dissolution, the pre-dissolution solution can be diluted to form the required concentration of, for example 8.3 mg/ml. Dilution may involve ×1, ×2, ×3 or more dilutions with WFI to obtain the target concentration. In embodiments, dilutions are carried out to achieve the desired concentration at appropriate molarity. By way of example, ×3 dilutions to add 2× the initial volume of organic acid may achieve 8.3 mg/mL in 0.1M organic acid (for example lactic acid).

During manufacture, there may be limited volume capacity for the dissolution step, and therefore lurbinectedin dissolution is advantageously achieved with limited organic acid. As such, using high molarity organic acid can achieve a high lurbinectedin concentration in a limited organic acid volume.

In embodiments, a multi-step compounding strategy is used to prepare lurbinectedin. Step 1 is the pre-dissolution step described above, for example: pre-dissolving partly crystalline lurbinectedin in lactic acid 0.31M at 25 mg/mL and diluting 3× with WFI to obtain the concentrated solution at 8.3 mg/mL in 0.1M lactic acid. To avoid precipitation of lurbinectedin, the remaining excipients should have acid pH when added to the compounding formulation. It has been found that the high concentration lurbinectedin solution can be mixed with a buffer solution at pH of 5.6 or less, for example between 4 to 5.6 or 4.2 to 5.6 without precipitation of lurbinectedin. As such, in Step 2, an organic buffer solution containing the bulking agent (eg disaccharide) may be prepared at suitable pH. By way of example, this may comprise the preparation of a 0.04 M sodium lactate buffer at pH of around 4.2 containing sucrose. In step 3, the solutions from step 1 and step 2 are combined to form the final bulk solution. The final bulk solution may be adjusted with WFI to achieve the final target weight. By way of example, in step 3, the 8.3 mg/mL lurbinectedin concentrated solution in 0.1M lactic acid with pH≈3 is diluted with 0.04M sodium lactate buffer pH≈4.2 containing sucrose. The final bulk solution composition after adjustment of WFI to final weight may be, by way of example, 0.5 mg/mL lurbinectedin in sodium lactate buffer pH=4+10% (w/v) sucrose. The present invention therefore identifies a compounding strategy to formulate partially crystalline lurbinectedin.

In one embodiment, the lyophilized composition comprises or consists of 4 mg of lurbinectedin, 800 mg of sucrose, 22.1 mg of lactic acid and 5.1 mg of sodium hydroxide. In some embodiments, the weight ratio in the lyophilized composition is between 0.4% and (w/w) of active compound, 96% to 98% (w/w) of sucrose, 2% to 3% (w/w) of lactic acid, and 0.5% to 0.7% (w/w) sodium hydroxide. In preferred embodiments, the weight ratio in the lyophilized composition is 0.5% (w/w) active compound, 96.2% (w/w) sucrose, 2.7% (w/w) lactic acid, and 0.6% (w/w) sodium hydroxide. The lyophilized formulation contains about 0.25 mmol of lactate ion for 4 mg of lurbinectedin. When reconstituted to 8 ml in the vial, the resulting solution is 0.5 mg/ml lurbinectedin, 0.03M sodium lactate buffer, 10% w/v sucrose at about pH 4.0 (range of pH 3.5 to 4.5, preferably 3.8 to 4.5).

The lyophilized material is usually present in a vial which contains a specified amount of lurbinectedin. Preferably the lyophilized composition of lurbinectedin is provided in a 30 mL vial. The specified amount of lurbinectedin in a lyophilized composition can be from between 0.2 to 5 mg, or about 1 mg, about 2 mg, about 3 mg, or about 4 mg. The specified amount of lurbinectedin in a lyophilized composition is preferably 4 mg. In lyophilized embodiments, the composition contains between 0.4% and by weight of lurbinectedin, preferably it is 0.5%.

It is necessary to ensure the lurbinectedin is sterile and is aseptically filled into vials. This is critical for parenteral drugs. According to embodiments of the present invention, terminal sterilization by heat or gamma irradiation are not used to avoid degradation of lurbinectedin. Instead, according to embodiments of the present invention, a sterilization filtration of the bulk lurbinectedin solution is carried out before aseptic vial filling. In embodiments, the filter may be filters such as PVDF or PES. In embodiments the filter may be a 0.2 μm filter.

### Storage of Pharmaceutical Lurbinectedin Formulations

Embodiments of this invention also provide a method of storing a lyophilized lurbinectedin composition, wherein the lyophilized lurbinectedin composition is manufactured from a solid-state form as disclosed herein—Form B. The use of solid state forms of the present invention may lead to advantageous storage properties as further discussed below. As discussed herein, using lurbinectedin as defined in the present invention can lead to advantages, including better control of impurities and/or degradation products.

It is necessary to ensure the lurbinectedin is stable during at least 24 months. The lurbinectedin lyophilized formulations are storage stable such that after prolonged storage at 5° C.±3° C., the lurbinectedin retains its therapeutic effectiveness and exhibits minimal chemical degradation (e.g., degradation is minimized and within acceptable tolerance; for example, the impurity and degradation products profile of the lurbinectedin, amount of each impurity and degradation product, lurbinectedin content, as determined by HPLC analysis, are substantially the same before and after prolonged storage).

In embodiments, the lyophilized lurbinectedin compositions of the present disclosure minimize the amount of a lurbinectedin degradation product resulting from deacetylation of lurbinectedin (“Impurity D”) when the composition is stored for prolonged times (e.g., at least 24 months). In some embodiments, the amount of impurity D present is less than 0.3%, 0.4%, 0.5%, 0.6%, 0.7% or 0.8% wt/wt of the total lurbinectedin weight in the formulation after prolonged storage at 5° C.±3° C. Impurity B, D and G have the following structures:

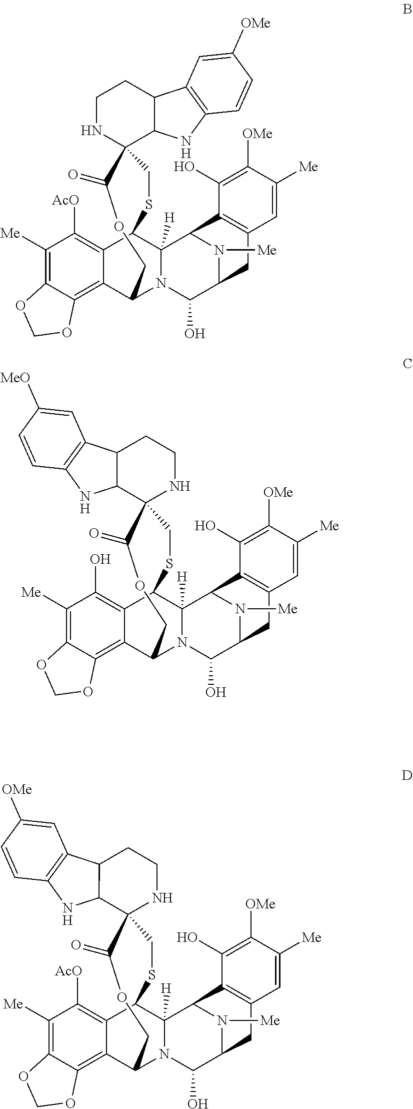

In a preferred embodiment the method of storing a lyophilized lurbinectedin composition comprises storing a lyophilized composition comprising 4 mg lurbinectedin; lactate buffer; and a disaccharide at a temperature of 5° C.±3° C. for at least 24 months, wherein the lyophilized composition is formulated such that reconstitution with 8 mL of water will yield a solution having a pH of 3.5 to 4.5 and a lurbinectedin concentration of 0.5 mg/ml and wherein after the at least 24 months storage, the amount of Impurity D present in the composition is not more than 0.8% wt./wt. of the total lurbinectedin weight. In some embodiments, the lyophilized lurbinectedin composition is stored at a temperature of 5° C.±3° C. for, or for at least, 24 months, 30 months, 36 months, 42 months, 48 months or 60 months, wherein after 24 months, 30 months, 36 months, 42 months, 48 months or 60 months of storage, the amount of a lurbinectedin degradation product Impurity D present in the composition is not more than 0.8% wt./wt. of the total lurbinectedin weight. In some embodiments, the amount of Impurity D present in the composition after storage at about 5° C.±3° C. for 60 months is not more than 0.8% wt./wt, or is less than 0.7% wt./wt., less than 0.6% wt./wt., less than 0.5% wt./wt., or less than 0.4% wt./wt. of the total lurbinectedin weight. In one embodiment, the amount of lurbinectedin degradation product Impurity D present in the composition is not more than 0.8% wt./wt. of the total lurbinectedin weight after at least 36 months of storage. In some embodiments, the total % impurities and degradation products (as % area) after storage at about 5° C.±3° C. for 24 months, 30 months or 36 months is not more than 0.6%, 0.7%, 0.8% 0.9% or 1.0% (% area). In some embodiments, the initial amount of Impurity D present in the composition (i.e., one day of lyophilization) is less than 0.4% wt./wt. of the total lurbinectedin weight. In some embodiments, the initial amount of Impurity D present in the composition is at least 0.05% wt./wt. or at least 0.1% wt./wt. of the total lurbinectedin weight. In some embodiments, the initial amount of Impurity D present in the composition is not more than 0.8% wt./wt., not more than 0.5% wt./wt. or not more than 0.1% wt./wt. of the total lurbinectedin weight. In some embodiments, after storage at about 5° C.±3° C. for 24 months, 30 months, 36 months, 48 months or 60 months the stable, lyophilized, lurbinectedin formulation shows negligible degradation of lurbinectedin assay content, for example, a decrease in the amount of lurbinectedin as compared to the amount of lurbinectedin within 1.0%, 0.5%, or 0.2% of the total amount of lurbinectedin as compared to the bulk solution from which the formulation is made.

Accordingly, provided are stable, lyophilized lurbinectedin formulations comprising a buffer derived from an organic acid (e.g., an organic carboxylic acid buffer, such as, succinate, citrate, acetate or lactate buffer) at a molar ratio of buffer to lurbinectedin of about 48, including the molar ratio 52 to 46, 54 to 44, 50 to 48, 52 to 58, or the molar ratio 51 to 48, and sucrose as a bulking agent, which, when reconstituted in 8 mL of water has a pH of about 4.0, including pH 3.5-4.5 or pH 3.8-4.1, which comprises Impurity D at no more than 0.8% wt/wt, or is less than 0.7% wt./wt., less than 0.6% wt./wt., less than 0.5% wt./wt., or less than 0.4% wt./wt of the total weight of lurbinectedin and, preferably, the Impurity D does not increase to more than 0.8% wt/wt of the total weight of lurbinectedin after storage at 5° C.±3° C. for 12 months, 24 months, 30 months, 36 months, 48 months or 60 months; or storage at 25° C./60% RH for 3 months, 6 months, 9 months, 12 months or 18 months; or 40° C./60% RH for 1 month, 3 months, 6 months or 12 months. In these embodiments, the lurbinectedin is 95 to 105%, or 97 to 103% of 4 mg lurbinectedin or of the amount of lurbinectedin by assay at day 1.

Also provided are methods of reducing lurbinectedin degradation in a lyophilized formulation by incorporating a buffer derived from an organic acid, preferably a lactate or succinate buffer, in the lyophilized formulation with the lurbinectedin such that the Impurity D in the formulation does not exceed 0.5% wt/wt, 0.6% wt/wt, 0.7% wt/wt or 0.8% wt/wt of the total lurbinectedin weight after storage at 5° C.±3° C. for 12 months, 24 months, 30 months, 36 months, 48 months or 60 months; or storage at 25° C./60% RH for 3 months, 6 months, 9 months, 12 months or 18 months; or 40° C./60% RH for 1 month, 3 months, 6 months or 12 months, particularly when the amount of lurbinectedin is 95 to 105%, or 97 to 103% of 4 mg lurbinectedin or of the amount of lurbinectedin by assay at day 1.

Other impurities or degradation products that may be minimized in the storage of the stable, lyophilized lurbinectedin formulation may be the degradation products with the following relative retention time on the commercial HPLC method: rrt 0.68, rrt 0.80, rrt 1.11 (Impurity G), and rrt 1.12.

In further embodiments, the total residual water content for the lyophilized lurbinectedin formulation is not more than 3% (w/w), preferably not more than 1.5% (w/w), preferably not more than 1% (w/w), is preferably between 0.5-0.7% (w/w).

Embodiments of this invention further provide a pharmaceutical product comprising a vial containing a lyophilized lurbinectedin composition. In a preferred embodiment, the pharmaceutical product comprises a vial containing a lyophilized composition consisting of 4 mg lurbinectedin; 22.1 mg lactic acid; 5.1 mg sodium hydroxide (or, including, about 0.25 mmol lactate); and 800 mg sucrose; and a label affixed to the vial comprising an expiration date that is at least 48 months from the date of manufacture. In some embodiments, the label affixed to the vial comprises an expiration date that is at least 24 months, at least 30 months, at least 36 months, at least 42 months, or at least 48 months from the date of manufacture. In some embodiments, the vial has a size of 30 mL to 50 mL, such as 30 mL, 35 mL, 40 mL, 45 mL, or 50 mL. In a preferred embodiment, the vial is a 30 mL vial. A vial size of 30 mL is optimized to overcome limitations of larger vial sizes which lead to production capacity reduction due to reduced freeze dryer capacity and also adequate extractable volumes due to size. A vial size of 30 mL overcomes both of these limitations.

As described herein, the solid-state forms of lurbinectedin as disclosed in the present invention may be used in compositions of the present invention. The compositions may be pre-lyophilisation compositions. The solid-state forms of lurbinectedin as disclosed in the present invention may be used to manufacture compositions of the present invention. The lurbinectedin may comprise Form B. The amount of Form B as disclosed herein may vary and can be considered a crystalline mixture (partially crystalline). Form B as disclosed herein may be used in the manufacturing process to prepare lyophilized bulk product. In some embodiments, the crystalline mixture may comprise other crystalline lurbinectedin (e.g. non form-B crystalline lurbinectedin).

In further embodiments, the present invention relates to a pharmaceutical composition comprising lurbinectedin manufactured using Form B of lurbinectedin and a pharmaceutically acceptable carrier. The pharmaceutical composition may no longer contain any Form B lurbinectedin, however the composition manufacturing process utilized at least some Form B in one or more steps. In further embodiments, the present invention relates to Form B of lurbinectedin for use in the manufacture of a pharmaceutical composition comprising lurbinectedin. In yet further embodiments, the present invention relates to the use of Form B of lurbinectedin in the manufacture of a pharmaceutical composition comprising lurbinectedin. In yet further embodiments, the present invention relates to Form B of lurbinectedin for use as a medicament. Again, Form B may no longer be present in the final composition but may be utilized during manufacturing. In yet further embodiments, the present invention further provides a method of treating any mammal, notably a human, affected by cancer which comprises administering to the affected individual a therapeutically effective amount of Form B of lurbinectedin or of a pharmaceutical composition comprising Form B of lurbinectedin and a pharmaceutically acceptable carrier; or a pharmaceutical composition made from a process utilizing Form B of lurbinectedin.

In a further embodiment, the present invention relates to lurbinectedin having residual solvents of not more than 1%, 0.5%, 0.1% or substantially not detected. In a further embodiment, the present invention relates to lurbinectedin having a water content of above 1.6% w/w, or of 1.7-5% w/w. In a further embodiment, the present invention relates to lurbinectedin having a water content of not more than 5%, 4% or 3% w/w.

The present invention encompasses lurbinectedin comprising at least a detectible amount of Form B, up to 1% w/w Form B, up to 5% w/w Form B, up to 10% w/w Form B, up to 20% w/w Form B, up to 30% w/w Form B, up to 40% w/w Form B, up to 50% w/w Form B, up to 60% w/w Form B, up to 70% w/w Form B, up to 80% w/w Form B, up to 90% w/w Form B, up to 95% w/w Form B, up to 98% w/w Form B, or be substantially pure Form B. In an embodiment, partially crystalline lurbinectedin as described herein may comprise at least a detectible amount of Form B, up to 1% w/w Form B, up to 5% w/w Form B, up to 10% w/w Form B, up to 20% w/w Form B, up to 30% w/w Form B, up to 40% w/w Form B, up to 50% w/w Form B, up to 60% w/w Form B, up to 70% w/w Form B, up to 80% w/w Form B, up to 90% w/w Form B, up to 95% w/w Form B, up to 98% w/w Form B, or be substantially pure Form B. w/w is intended to mean the amount of lurbinectedin which is in the Form B state. As such, purely by way of example, 50% w/w means the lurbinectedin API comprises 50% by weight Form B and 50% by weight another form, for example amorphous Form A.

In an embodiment, the present invention relates to pharmaceutical compositions comprising Form B of lurbinectedin and a pharmaceutically acceptable carrier or manufactured from lurbinectedin comprising Form B. The lurbinectedin used in the compositions or used during the manufacture of the compositions may comprising lurbinectedin comprising at least a detectible amount of Form B, up to 1% w/w Form B, up to 5% w/w Form B, up to 10% w/w Form B, up to 20% w/w Form B, up to 30% w/w Form B, up to 40% w/w Form B, up to 50% w/w Form B, up to 60% w/w Form B, up to 70% w/w Form B, up to 80% w/w Form B, up to 90% w/w Form B, up to 95% w/w Form B, up to 98% w/w Form B, or be substantially pure Form B.

Partially crystalline lurbinectedin as disclosed herein may in embodiments comprise at least a detectible amount of Form B, up to 1% w/w Form B, up to 5% w/w Form B, up to 10% w/w Form B, up to 20% w/w Form B, up to 30% w/w Form B, up to 40% w/w Form B, up to 50% w/w Form B, up to 60% w/w Form B, up to 70% w/w Form B, up to 80% w/w Form B, up to 90% w/w Form B, up to 95% w/w Form B, up to 98% w/w Form B, or be substantially pure Form B. In alternative embodiments, other non-Form B crystalline lurbinectedin may form partially crystalline lurbinectedin at the same w/w amounts.

The partially crystalline lurbinectedin as disclosed herein may be used to form pharmaceutical compositions according to the present invention. Accordingly, in embodiments, partially crystalline lurbinectedin is used in the manufacture of a bulk lurbinectedin solution which is thereafter lyophilized to form the lyophilized lurbinectedin formulation. The partially crystalline lurbinectedin may comprise Form B as disclosed herein. Thus, in embodiment where reference is made to partially crystalline lurbinectedin it is intended to mean at least some Form B.

Although the partially crystalline lurbinectedin may not be present in the final dosage form (due to the dissolution and subsequent lyophilisation steps), it nevertheless may affect the properties of the final dosage form. By way of example, using partially crystalline lurbinectedin can reduce and/or simplify the total impurities including degradation products. Characteristic impurity profiles may demonstrate the use of partially crystalline lurbinectedin during manufacture. According to an embodiment, the total degradation products in the final lyophilized product may be not more than (NMT) 1.9%, 1.8%, 1.7%, 1.6%, 1.5%, 1.4%, or 1.3%. In a preferred embodiment, the total degradation products are NMT than 1.3%. According to a further embodiment, the final lyophilized product comprises NMT 0.8% of impurity D. According to a further embodiment, the final lyophilized product comprises NMT 0.3% of any unspecified impurity.

Using partially crystalline lurbinectedin may also advantageously control residual solvents. In an embodiment, the lurbinectedin comprises not more than 0.2% residual solvents, preferably not more than 0.1% residual solvents, preferably residual solvents are substantially not detected.

In an embodiment, the partially crystalline lurbinectedin used in the manufacture of the compositions disclosed herein may have an assay (%) in the range 94.0-102.0% and an impurities level lower than 1.0%. Specified impurities and their limits may be are impurity B (≤0.20%), impurity D (≤0.50%) and/or impurity G (≤0.50%). Any other individual non-specified impurity may have a limit of ≤0.20%. In particular embodiments, the % wt/wt of Impurity D relative to lurbinectedin does not increase by more than 0.1%, 0.2% or 0.3% wt/wt upon storage of reconstituted or diluted solution for 24, 48 or 72 hours at either room temperature (i.e., about 23° C.)/light or under refrigerated (5° C.±3° C.) conditions.

In particular embodiments according to the present invention, lyophilized composition according to the present (utilizing the solid state form invention as disclosed herein) comprise less than about 0.3% of Impurity D (w/w based on lurbinectedin) when the composition is packaged, and wherein upon storage at about 5 degrees C. for about 24, 36 or 48 months the composition comprises less than about 0.8% of Impurity D (w/w based on lurbinectedin).

### EXAMPLES

### Abbreviations

| | |
|---|---|
| DSC | Differential scanning calorimetry |
| DVS | Dynamic vapor sorption |
| XRPD | X-ray powder diffractograms |
| TG-FTIR | Thermogravimetry coupled with Fourier transformed |
| | infrared spectroscopy |
| r.h. | Relative humidity |

The X-ray powder diffractograms (XRPD) were obtained with a Stadi P diffractometer (Stoe & Cie GmbH) in transmission geometry, equipped with a curved Ge-crystal monochromator, a Cu-Kα1 radiation source and a Mythen1K Detector in step scan detector mode. The pattern was recorded at a tube voltage of 40 kV, tube current of 40 mA, applying a stepsize of 0.02° 2-theta with 12 seconds per step in the angular range of 1.5° to 50.5° 2-theta. The detector step was 1° 2-theta. A typical precision of the 2-theta values is in the range of about ±0.2° 2-theta. Thus a diffraction peak that appears at 5.0° 2-theta can appear between 4.8 and 5.2 2-theta on most X-ray diffractometers under standard conditions.

TG-FTIR experiments were conducted with a Thermo-Microbalance TG-209 (Netzsch) equipped with a FT-IR Spectrometer Vector 22 (Bruker) using Al crucible (open or with microhole) under N₂ atmosphere with a heating range between 25 and 250° C. and a heating rate of 10° C./min.

DSC experiments were carried out with a Perkin Elmer DSC 7 using closed Au crucibles with a heating range between −50 to 250° C. and a heating rate of 10 or 20° C./min.

DVS experiments were carried out with a Projekt Messtechnik SPS 11-100n multi-sample water vapor sorption analyzer. The sample was allowed to equilibrate at 50% r.h. before starting a pre-defined humidity program. The program was:
2 h at 50% r.h.
50 to 0% r.h. (5%/h)
0 h at 0% r.h.
0 to 95% r.h (5%/h)
5 h at 95% r.h.
95 to 50% (5%/h)
2 h at 50% r.h.

### Example 1. Manufacture of Amorphous Form a of Lurbinectedin

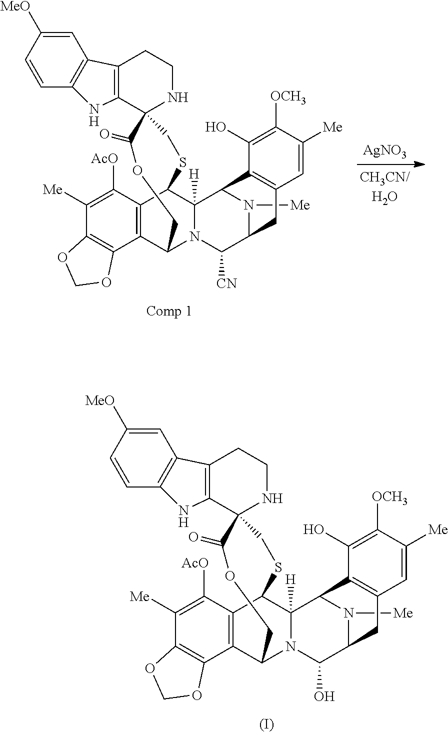

Form A of lurbinectedin was obtained following the procedure described in WO 03/014127.

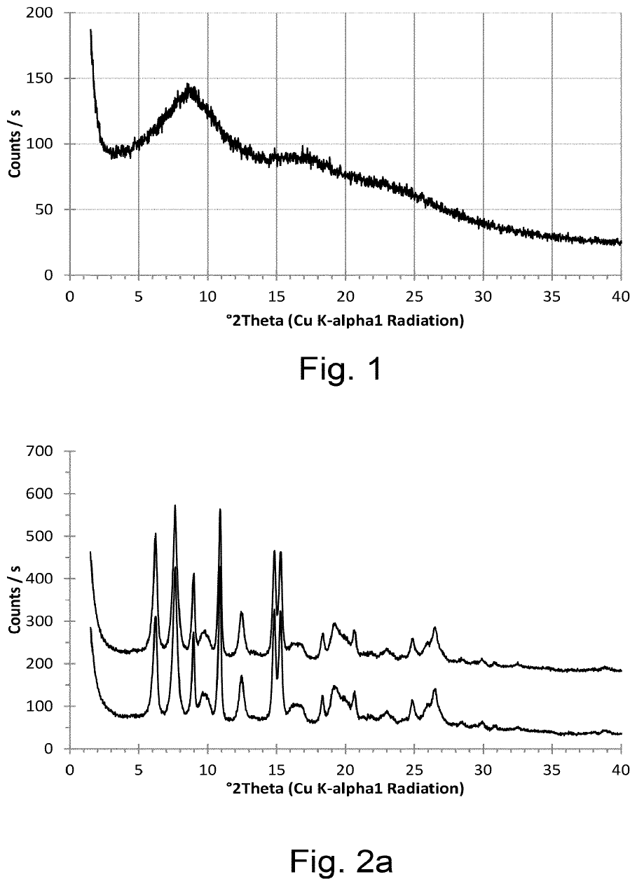

The XRPD pattern of form A of lurbinectedin confirmed that this form is amorphous. See FIG. 1.

Several batches of form A of lurbinectedin were manufactured by this method. The analytical results of five of them are shown in Table 1.

TABLE 1

| | | Batch | | | | |
|---|---|---|---|---|---|---|
| | | P01 | P02 | P03 | P04 | R05 |
| Impurities (% Area) | Total | 0.3 | 0.4 | 0.3 | 0.2 | 0.4 |
| Residual Solvents | Total | 1.4 | 1.9 | 1.9 | 2.1 | 2.5 |
| (% w/w) | Acetonitrile | 0.01 | <LOQ | <LOQ | <LOQ | <LOQ |
| | Dichloromethane | <LOQ | <LOQ | 0.01 | <LOQ | <LOQ |
| | Ethyl acetate | <LOQ | <LOQ | <LOQ | <LOQ | <LOQ |
| | Hexane | <LOQ | 0.01 | <LOQ | <LOQ | <LOQ |
| | Pentane | 0.2 | 0.2 | 0.3 | 0.5 | 0.3 |
| | Methanol | 1.1 | 1.7 | 1.6 | 1.6 | 2.2 |
| Water Content (% w/w) | | 0.9 | 1.2 | 1.6 | 1.6 | 1.1 |

ND: not detected
LOQ: limit of quantification

### Impurity Profile

Table 2 shows the impurity profile of several batches of form A lurbinectedin.

TABLE 2

| Impurity | | | | | | | | | | | | | |
|---|---|---|---|---|---|---|---|---|---|---|---|---|---|
| RRT | Form A | | | | | | | | | | | | |
| approx. | F02 | G01 | K02 | K03 | K04 | M01 | M02 | M03 | P01 | P02 | P03 | P04 | R05 |
| 0.66-0.69 | 0.19 | 0.06 | — | — | — | — | — | — | — | — | — | — | — |
| 0.72-0.76 | 0.06 | 0.11 | 0.09 | 0.07 | 0.10 | 0.10 | 0.08 | 0.12 | 0.10 | 0.09 | 0.09 | 0.09 | 0.10 |
| 0.90-0.92 | — | — | — | — | — | — | — | — | — | — | — | — | — |
| 1.10-1.11 | 0.17 | 0.50 | 0.13 | 0.17 | 0.20 | 0.08 | 0.10 | 0.11 | 0.15 | 0.09 | 0.08 | — | 0.09 |
| 1.12 | — | — | — | — | — | — | — | — | — | — | 0.07 | — | — |
| 1.27-1.34 | 0.12 | — | — | — | — | — | — | — | — | — | — | — | — |
| 1.29-1.30 | — | — | 0.07 | 0.37 | 0.11 | 0.10 | 0.17 | 0.10 | 0.09 | 0.06 | 0.09 | 0.10 | 0.05 |
| 1.30-1.32 | 0.09 | 0.13 | 0.19 | 0.08 | 0.08 | 0.18 | 0.08 | 0.11 | — | 0.13 | — | — | — |
| 1.28-1.37 | — | — | — | — | — | — | 0.08 | — | — | — | — | — | — |
| 2.37 | — | — | — | — | — | — | — | — | — | — | — | — | 0.13 |
| 2.41-2.52 | 0.07 | — | — | — | — | — | — | — | — | — | — | — | — |
| 2.85 | 0.07 | — | — | — | — | — | — | — | — | — | — | — | — |

RRT—Relative Retention Time

### Example 2. Manufacture of Form B of Lurbinectedin

Crude lurbinectedin (10 g), which was obtained as described in Example 1, was dissolved in aqueous HCl (0.1 M, 390 mL). The aqueous solution was washed with CH₂Cl₂ (2×335 mL) and with n-pentane (1×335 mL) and treated with an aqueous solution of NH₄Cl/NH₄OH (prepared by dissolving 17.5 g of NH₄Cl and 20 mL of NH₄OH in 250 mL of water, 68 mL) to precipitate form B of lurbinectedin, that was filtered, washed with water and dried under vacuum to give 7.5 g, 9.45 mmol, yield 81% of form B of PM01183.

### Batch Analysis

Several batches of form B of lurbinectedin were manufactured by this method. The analytical results of ten of them are shown in Table 3.

TABLE 3

| | | Batch | | | | | | | | | |
|---|---|---|---|---|---|---|---|---|---|---|---|
| | | 1711182-2 | 1711189-2 | 1799069 | 1924129-LT | 1924128-LT | R01 | R02 | R03 | R04 | P05 |
| Total Impurities | | 0.3 | 0.3 | 0.4 | 0.4 | 0.4 | 0.3 | 0.3 | 0.3 | 0.4 | 0.3 |
| (% Area) | | | | | | | | | | | |
| Residual | Total | 0.0 | 0.0 | 0.0 | 0.0 | 0.0 | 0.0 | 0.0 | 0.0 | 0.0 | 0.0 |
| Solvents | Acetonitrile | <LOQ | <LOQ | <LOQ | <LOQ | <LOQ | <LOQ | <LOQ | <LOQ | <LOQ | <LOQ |
| (% w/w) | CH₂Cl₂ | <LOQ | <LOQ | <LOQ | <LOQ | <LOQ | <LOQ | <LOQ | <LOQ | <LOQ | <LOQ |
| | AcOEt | NA | NA | NA | NA | NA | <LOQ | NA | NA | NA | <LOQ |
| | Hexane | NA | NA | NA | NA | NA | <LOQ | NA | NA | NA | <LOQ |
| | Pentane | <LOQ | <LOQ | <LOQ | <LOQ | <LOQ | <LOQ | <LOQ | <LOQ | <LOQ | <LOQ |
| | Methanol | <LOQ | <LOQ | <LOQ | <LOQ | <LOQ | <LOQ | NA | <LOQ | <LOQ | <LOQ |

| | | | | | | | | | | |
|---|---|---|---|---|---|---|---|---|---|---|
| Water Content (% w/w) | 2.4 | 2.5 | 2.0 | 4.1 | 3.0 | 1.9 | 2.0 | 1.7 | 2.6 | 2.1 |

LOQ: limit of quantification
NA: not analyzed

An additional advantage of form B of lurbinectedin over form A of lurbinectedin is the absence of residual solvents.

### Impurity Profile

Table 4 shows the impurity profile (% area) of several batches of form B of lurbinectedin

TABLE 4

| Impurity | Form B | | | | | | | | | |
|---|---|---|---|---|---|---|---|---|---|---|
| RRT | | | | | | | | 1924129- | 1924128- | |
| aprox | R01 | R02 | R03 | R04 | 1711182-2 | 1711189-2 | 1799069 | LT | LT | P05 |
| 0.66-0.69 | — | — | 0.05 | 0.05 | — | 0.05 | — | 0.07 | 0.06 | — |
| 0.72-0.76 | 0.22 | 0.28 | 0.22 | 0.26 | 0.34 | 0.30 | 0.31 | 0.24 | 0.25 | 0.24 |
| 0.90-0.92 | — | — | — | — | — | — | — | 0.08 | 0.07 | — |
| 1.10-1.11 | 0.09 | — | — | 0.07 | — | — | 0.06 | — | — | 0.07 |
| 1.12 | — | — | — | — | — | — | — | — | — | — |
| 1.27-1.34 | — | — | — | — | — | — | — | — | — | — |
| 1.29-1.30 | — | — | — | — | — | — | — | — | — | — |
| 1.30-1.32 | — | — | — | — | — | — | — | — | — | — |
| 1.28-1.37 | — | — | — | — | — | — | — | — | — | — |
| 2.41-2.52 | — | — | — | — | — | — | — | — | — | — |
| 2.85 | — | — | — | — | — | — | — | — | — | — |

RRT—Relative Retention Time

A comparison between the impurity profiles of forms A and B of lurbinectedin clearly shows that form B of lurbinectedin consistently presents fewer impurities than form A of lurbinectedin.

### Solid State Characterization

Form B of lurbinectedin was characterized by XRPD, IR, TG-FTIR, DSC and DVS.

The XRPD pattern of several batches of Form B of lurbinectedin confirmed that this form is partly crystalline (broad peaks, amorphous background) and that the process for its manufacture is reproducible. See FIGS. 2a and 2b. XRPD angles 2-theta and their relative intensities of two batches of form B of lurbinectedin are shown in Table 5.

TABLE 5
XRPD angles 2-theta, relative intensities of two batches of
Form B of lurbinectedin

| Batch 1924128-LT | | Batch 1924129-LT | |
|---|---|---|---|
| Angle | Relative | Angle | Relative |
| [2-theta] | intensity[%] | [2-theta] | intensity[%] |
| 6.2 | 84 | 6.2 | 73 |
| 7.6 | 100 | 7.7 | 100 |
| 9.0 | 62 | 9.0 | 64 |
| 9.8 | 31 | 9.6 | 30 |
| 10.9 | 98 | 10.9 | 100 |
| 12.4 | 41 | 12.4 | 40 |
| 14.9 | 75 | 14.8 | 77 |
| 15.3 | 74 | 15.3 | 76 |
| 18.4 | 29 | 18.3 | 29 |
| 19.2 | 34 | 19.2 | 35 |
| 20.7 | 31 | 20.7 | 32 |
| 24.9 | 26 | 24.9 | 27 |
| 26.5 | 32 | 26.5 | 33 |

TG-FTIR indicates degradation above 150° C. for form B of lurbinectedin. A release of 2.6% of water was detected. See FIG. 3.

Estimation of the amorphous content by DSC was not possible. Degradation was observed to begin above 130° C., see FIG. 4. A glass transition temperature or melting point was not detected.

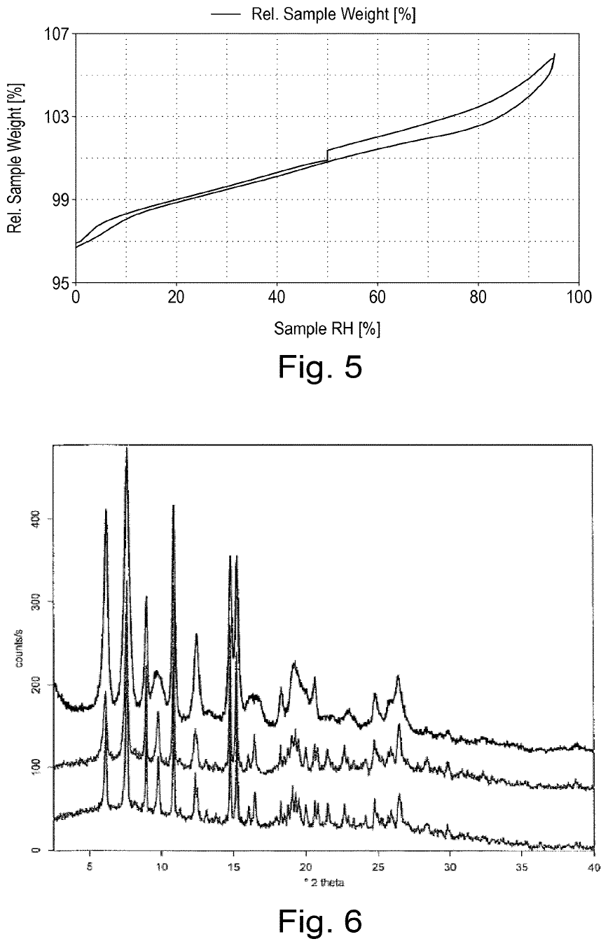

DVS indicates a continuous water uptake and release with no steps and almost no hysteresis. This is due to the partly amorphous character of form B. The sample is not deliquescent. A mass change of Δm (50 to 96% r.h.)≈4% was observed, indicating that form B of lurbinectedin is hygroscopic. Upon lowering the relative humidity again, the water content decreased and nearly returned to the original mass, see FIG. 5.

### Relative Stability of Form B of Lurbinectedin

Three 1:1 mixtures of forms A and B of lurbinectedin (15 mg each) were prepared and suspended in water (1 mL). Samples were taken after 6 and 24 hours. The powder patterns after 6 and 24 hours agree with that of the form B starting material. See FIG. 6. Both patterns after phase equilibration show sharper peaks and higher peak resolution. These are indications of improved crystallinity. A quantification of the amorphous content was not possible with the available data.

### IR

IR spectra were obtained for form A of lurbinectedin, shown in FIG. 7a, and for form B of lurbinectedin, shown in FIG. 7b. The correlation factor between the IR of three batches of form B of lurbinectedin and the IR of form A of lurbinectedin varies from 0.81 to 0.86. On the other hand, the correlation factor of several IR spectra of form A of lurbinectedin varies from 0.97 to 0.99.

### Example 3. Electrostatic Charge Measurements in Air

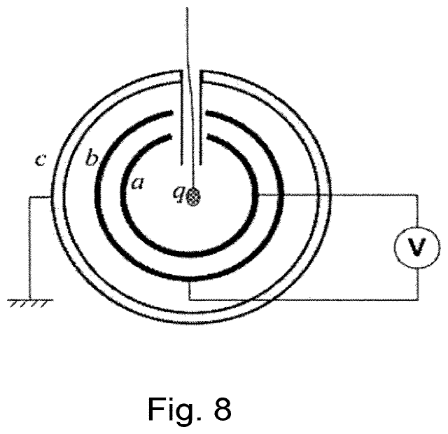

The electrostatic charges of two batches of form A of lurbinectedin (Batches P04 and R05) and of form B of lurbinectedin (Batch 1924129-LT and 1924128-LT) have been measured using a Faraday cage (See FIG. 8) constructed of stainless steel with concentric spheres with ratios 10, 15 and 20 cm.

This technique consists in placing the sample to be measured (q) in the interior of the inner sphere (a) and measuring the difference of potential induced between sphere (a) and another conductor of reference, sphere (b). The external sphere (c) is grounded in order to shield the system. The measurements of the difference of potential were carried out with a precision electrometer (Keithley 617, resolution 10fC.

Measurements were carried out under controlled atmosphere of dry nitrogen in order to avoid the effect of ambient humidity on the electrostatic charge of the samples.

Samples were introduced in glass capsules using non-conductor instrumental to avoid loses of electrostatic charge.

The capsules loaded with the samples were introduced in the Faraday cage through a grounded conductor tube to avoid parasitic static charges in the glass capsule. The entry and removal of the capsules was done with a computer-controlled servo engine, in order to ensure a constant rate of introduction and removal of the capsules in each measurement to minimize the creation of static charges due to friction of the insulator elements.

### Results:

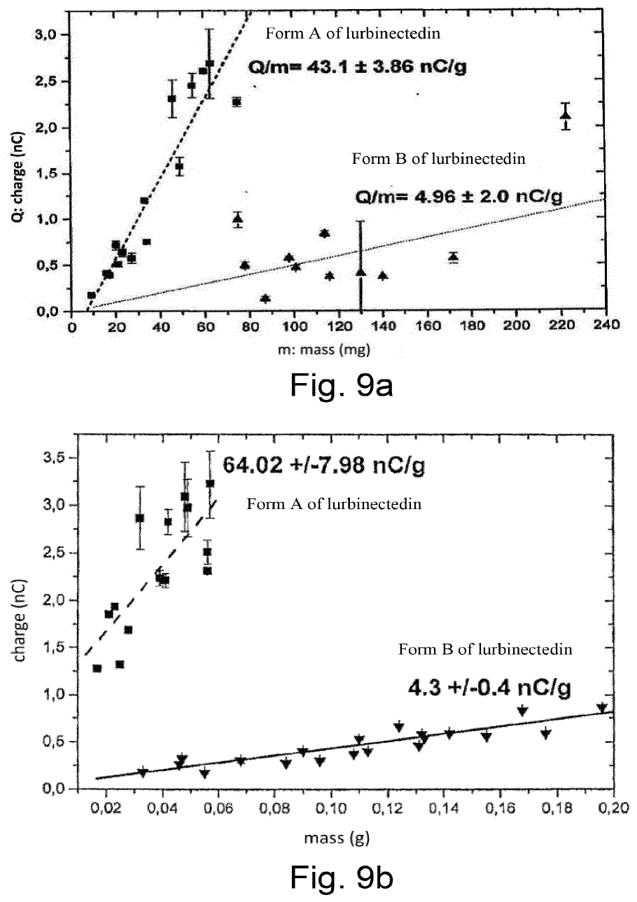

Several measurements with different amounts of material were carried out for each batch of each form of lurbinectedin. Before loading the capsules, they were washed and their remaining static charge was measured in order to correct the levels. Each sample was introduced and removed five times and, after each introduction, several consecutive measurements were taken in order to average any possible drift effect. FIGS. 9a and 9b summarized the results of such measurement for each pair of batches of forms A and B of lurbinectedin.

The measured charge Q increases with the amount of analyzed material. Both forms of lurbinectedin have a positive electrostatic charge. Form A of lurbinectedin has a total static charge considerably higher than form B of lurbinectedin. The data was fitted by lineal regression (dashed lines in FIGS. 9a and 9b) to obtain the charge density (Q/m) as the slope of the line. The extrapolation of the lineal regression to a mass of 0 mg represents the remnant electrostatic charge of the glass capsules, and does not affect the value of Q/m. The results of this regression and the dispersion of charge density are summarized in Table 6. All ranges are given with a 95% confidence.

TABLE 6

| | | Average charge | Dispersion of charge |
|---|---|---|---|
| Form | Batch | density (Q/m) (nC/g) | density (Q/m) (nC/g) |
| A | P04 | 43.1 ± 3.86 | 7.6 ± 2.8 |
| | R05 | 64.02 ± 7.98 | 15.23 ± 5.64 |
| B | 1924129-LT | 4.96 ± 2.0 | 3.4 ± 1.4 |
| | 1924128-LT | 4.3 ± 0.4 | 1.01 ± 0.3 |

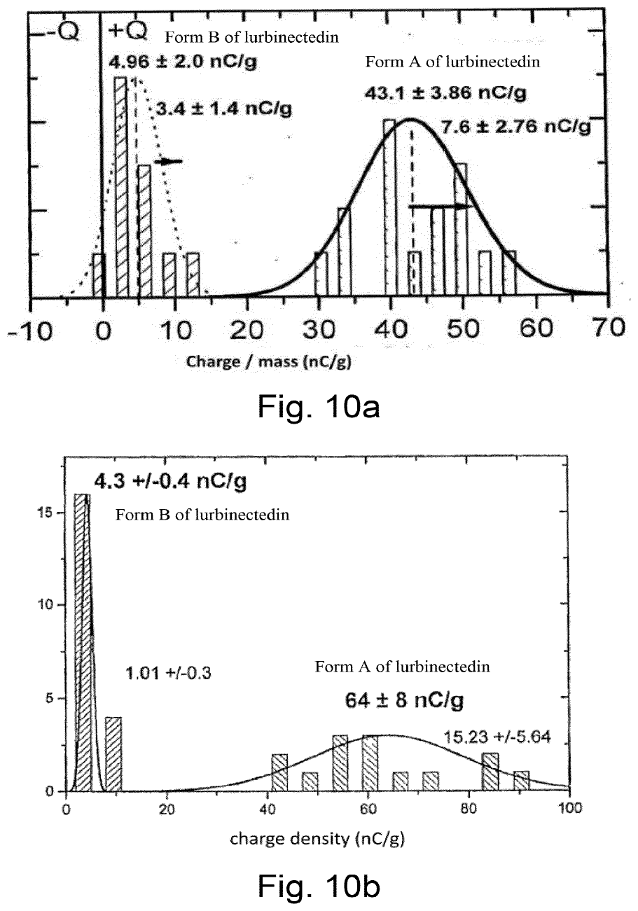

FIGS. 10a and 10b show the distribution of charge density for each pair of batches of forms A and B of lurbinectedin.

### CONCLUSIONS

Form B of lurbinectedin has an average charge density one order of magnitude lower than form A of lurbinectedin. This difference in triboelectrization has been demonstrated using two different batches of each form.

### Example 4 Process for the Manufacture of a Pharmaceutical Composition Using Form B or PM01183 as Starting Material

Form B of lurbinectedin was dissolved in a concentrated lactic acid solution (0.31 M) at a concentration of 25 mg/ml. Then, this solution was diluted with water of injection (WFI) to a lactic acid solution (0.1 M) containing PM01183 at a concentration of 8.33 mg/ml.

This solution was then added under stirring into a sucrose/buffer solution (pH=4.2) previously prepared, composed of lactic acid (3.7 mg/ml), sodium hydroxide (1.1 mg/ml) and the bulking agent, sucrose (167.7 mg/ml). If required, the mixed solution will be adjusted to pH=4.0 with lactic acid solution or sodium hydroxide solution.

Then, the bulk solution was brought to final volume or weight (considering a density value of 1.04 g/cc), generating the final bulk solution (0.5 mg/ml lurbinectedin, 2.76 mg/ml lactic acid, 0.64 mg/ml NaOH, 100 mg/ml sucrose).

The bulk solution was then filtered through sterilizing PVDF filters (0.22 lam) and filled into 30 ml glass vials at 8 ml/vial.

The vials were lyophilised according to a cycle detailed in Table 7.

TABLE 7

| Step (conditions) | Time |
|---|---|
| Freezing, −5° C. | 1.5 h |
| Freezing, −40° C. | 5.5 h |
| Primary drying (−25° C., 0.1-0.2 mb) | 60 h |
| Secondary drying (+25° C., maximum vacuum) | 30 h |
| Stoppering | NA |

NA: not applicable

After lyophilization, vials were sealed with flip-off seals and stored at +5° C.

1. A crystalline form of lurbinectedin of formula (I): 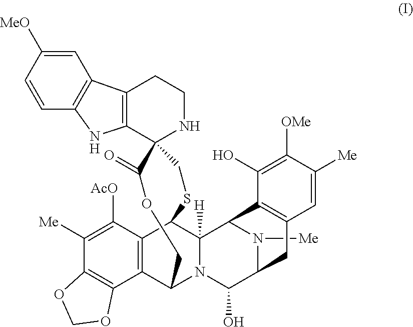 [SMILES: COc1ccc2[nH]c3c(c2c1)CCN[C@]31CS[C@@H]2c3c(OC(C)=O)c(C)c4c(c3[C@H](COC1=O)N1[C@@H]2[C@H]2c3c(cc(C)c(OC)c3O)C[C@@H]([C@@H]1O)N2C)OCO4]
characterized by an X-ray powder diffraction pattern using Cu-Kα1 radiation comprising three or more peaks at 2-theta angles selected from 6.2±0.2°, 7.6±0.2°, 9.0±0.2°, 10.9±0.2°, 14.9±0.2°, and 15.3±0.2°.

2. The crystalline form according to claim 1, wherein the X-ray powder diffraction pattern using Cu-Kα1 radiation comprising four or more peaks at 2-theta angles selected from 6.2±0.2°, 7.6±0.2°, 9.0±0.2°, 10.9±0.2°, 14.9±0.2°, and 15.3±0.2°.

3. The crystalline form according to claim 1, wherein the X-ray powder diffraction pattern using Cu-Kα1 radiation comprising five or more peaks at 2-theta angles selected from 6.2±0.2°, 7.6±0.2°, 9.0±0.2°, 10.9±0.2°, 14.9±0.2°, and 15.3±0.2°.

4. The crystalline form according to claim 1, wherein the X-ray powder diffraction pattern using Cu-Kα1 radiation comprises peaks at 2-theta angles of 6.2±0.2°, 7.6±0.2°, and 10.9±0.2°.

5. The crystalline form according to claim 2, wherein the X-ray powder diffraction pattern using Cu-Kα1 radiation comprises peaks at 2-theta angles of 6.2±0.2°, 7.6±0.2°, 10.9±0.2° and 14.9±0.2°.

6. The crystalline form according to claim 3, wherein the X-ray powder diffraction pattern using Cu-Kα1 radiation comprises peaks at 2-theta angles of 6.2±0.2°, 7.6±0.2°, ±0.2°, 14.9±0.2°, and 15.3±0.2°.

7. The crystalline form according to claim 1, wherein the X-ray powder diffraction pattern using Cu-Kα1 radiation comprises peaks at 2-theta angles of 6.2±0.2°, 7.6±0.2°, 9.0±0.2°, 10.9±0.2°, 14.9±0.2°, and 15.3±0.2°.

8. The crystalline form according to claim 7, wherein the X-ray powder diffraction pattern using Cu-Kα1 radiation further comprises peaks at 2-theta angles of 12.4±0.2°, 19.2±0.2° and 26.5±0.2°.

9. The crystalline form according to claim 8, wherein the X-ray powder diffraction pattern using Cu-Kα1 radiation further comprises peaks at 2-theta angles of 18.4±0.2°, and 24.9±0.2°.

10. The crystalline form according to claim 1, wherein the X-ray powder diffraction pattern using Cu-Kα1 radiation exhibits an X-ray powder diffraction pattern substantially the same as the X-ray powder diffraction patterns shown in FIG. 2a.

11. The crystalline form according to claim 1, further characterized by a TG-FTIR mass change to 150° C. of around 2-3%.

12. The crystalline form according to claim 1, further characterized by an average charge density of not more than 10 nC/g.

13. The crystalline form according to claim 1, further characterized by an average charge density of about 5±2 nC/g.

14. The crystalline form according to claim 1, further characterized by a dispersion of charge density between about 0.7 nC/g to less than 4.8 nC/g.

15. The crystalline form according to claim 1, further characterized by residual solvents of not more than 0.1%.

16. The crystalline form according to claim 1 prepared by a process comprising the steps of
a) preparing an acidic aqueous solution comprising lurbinectedin or a protonated form thereof; and
b) basifying the acidic aqueous solution with a base or buffer to precipitate the crystalline form of lurbinectedin.

17. The crystalline form according to claim 16; wherein the acidic aqueous solution is prepared by dissolving any form of lurbinectedin in an acidic water made from a pharmaceutically acceptable acid and wherein the acidic water has a pH of 1 to 4.

18. The crystalline form according to claim 17, wherein the pharmaceutically acceptable acid is HCl and the pH of the acidic aqueous solution in step (a) is 2 to 3.

19. The crystalline form according to claim 16, wherein the base is sodium carbonate, potassium carbonate, NH₄OH, lithium hydroxide, sodium hydroxide, potassium hydroxide, sodium hydrogen carbonate, or potassium hydrogen carbonate.

20. The crystalline form according to claim 16, wherein the buffer is a KH₂PO₄ buffer or a Na₂HPO₄ and citric acid buffer or an ammonium chloride buffer.

21. The crystalline form according to claim 16, wherein the acidic aqueous solution is basified in step (b) with ammonium chloride and ammonium hydroxide.

22. The crystalline form according to claim 16, wherein the process further comprises washing the acidic aqueous solution one or more times with a pharmaceutically acceptable, water-immiscible, polar solvent followed by washing the acidic aqueous solution one or more times with a pharmaceutically acceptable, water-immiscible, non-polar solvent before basifying the acidic aqueous solution.

23. The crystalline form according to claim 22, wherein the non-polar solvent is an alkane having 5 to 10 carbons.

24. The crystalline form according to claim 22, wherein the alkane has 5 to 7 carbons.

25. The crystalline form according to claim 22, wherein the alkane is n-pentane.

26. The crystalline form according to claim 22, wherein the polar solvent is chloroform, ethyl acetate or dichloromethane.

27. The crystalline form according to claim 26, wherein the polar solvent is dichloromethane.

28. The crystalline form according to claim 27, wherein the pH of the aqueous solution after step (b) is 8 to 11.

29. A composition comprising the crystalline form of lurbinectedin according to claim 1 and amorphous lurbinectedin.
